(19) United States
(12) Patent Application Publication
(10) Pub. No.: US 2006/0115810 A1
(43) Pub. Date: Jun. 1, 2006

(54) Microarrays of functional biomolecules, and uses therefor

(75) Inventors: Michael H. Cardone, Boston, MA (US); Ulrik Nielsen, Jamaica Plain, MA (US); Gavin MacBeath, Arlington, MA (US); James D. Marks, San Francisco, CA (US); Peter Sorger, Winchester, MA (US); Anthony Sinskey, Boston, MA (US)

Correspondence Address: WOLF GREENFIELD & SACKS, PC;FEDERAL RESERVE PLAZA, 600 ATLANTIC AVENUE, BOSTON, MA, 02210-2206, US

(73) Assignees: Massachusetts Institute of Technology, Cambridge, MA (US); The Regents of The University of California, Oakland, CA (US); President and Fellows of Harvard College, Cambridge, MA (US)

(21) Appl. No.: 11/239,652

(22) Filed: Sep. 29, 2005

Related U.S. Application Data
(62) Division of application No. 09/921,655, filed on Aug. 3, 2001, now abandoned.
(60) Provisional application No. 60/222,763, filed on Aug. 3, 2000.

(51) Int. Cl. C12Q 1/70; G01N 33/542; C12M 1/34; G01N 33/543; G01N 33/53

(52) U.S. Cl. 435/5; 436/518; 435/7.9; 435/287.2

(57) ABSTRACT

Disclosed are products and methods to facilitate the identification of compounds that are capable of interacting with biological macromolecules of interest, especially when such macromolecules are attached to a support surface in microarray. Aspects of the invention concern attachment chemistry, peptide labeling, antibody preparation, applications and so on.

### RELATED APPLICATIONS

This application is a divisional application of U.S. patent application Ser. No. 09/921,655, filed Aug. 3, 2001, which is based on and claims priority to U.S. Provisional Patent Application No. 60/222,763, filed on Aug. 3, 2000, the disclosures of which are hereby incorporated by reference.

### FIELD OF THE INVENTION

The present invention relates to the field of diagnostic and analytical chemistry, and particularly to devices for screening complex chemical or biological samples to identify, isolate or quantify components within a sample based upon their ability to bind to specific binding elements. The invention is particularly related to the production and use of arrays, preferably microarrays, of binding elements which are of biological significance or which bind to ligands of biological significance.

### BACKGROUND OF THE INVENTION

To construct high-density arrays of functional biomolecules for efficient screening of complex chemical or biological samples or large numbers of compounds, the binding elements need to be immobilized onto a solid support. A variety of methods are known in the art for attaching biological molecules to solid supports. See generally, Affinity Techniques, Enzyme Purification. Part B, Meth. Enz. 34 (ed. W. B. Jakoby and M. Wilchek, Acad. Press, N.Y. 1974) and Immobilized Biochemicals and Affinity Chromatography, Adv. Exp. Med. Biol. 42 (ed. R. Dunlap, Plenum Press, N.Y. 1974). Arenkov et al., for example, have described a way to immobilize proteins while preserving their function by using microfabricated polyacrylamide gel pads to capture proteins, and then accelerating diffusion through the matrix by microelectrophoresis (Arenkov et al. (2000), Anal Biochem 278(2):123-31). The patent literature also describes a number of different methods for attaching biological molecules to solid supports. For example, U.S. Pat. No. 4,282,287 describes a method for modifying a polymer surface through the successive application of multiple layers of biotin, avidin, describes a method for modifying a polymer surface through the successive application of multiple layers of biotin, avidin, and extenders. U.S. Pat. No. 4,562,157 describes a technique for attaching biochemical ligands to surfaces by attachment to a photochemically reactive arylazide. Irradiation of the azide creates a reactive nitrene that reacts irreversibly with macromolecules in solution, resulting in the formation of a covalent bond. The high reactivity of the nitrene intermediate, however, results in both low coupling efficiencies and many potentially unwanted products due to nonspecific reactions. U.S. Pat. No. 4,681,870 describes a method for introducing free amino or carboxyl groups onto a silica matrix, in which the groups may subsequently be covalently linked to a protein in the presence of a carbodiimide. In addition, U.S. Pat. No. 4,762,881 describes a method for attaching a polypeptide chain to a solid substrate by incorporating a light-sensitive unnatural amino acid group into the polypeptide chain and exposing the product to low-energy ultraviolet light.

There remains, however, a need for more efficient and easy-to-make array systems that identifies, isolates and/or quantifies components within complex samples, as well as to screen large numbers of compounds based upon their ability to bind to a variety of different binding partners.

### SUMMARY OF THE INVENTION

The present invention provides microarray assay systems where binding elements of interest are immobilized on a substrate and are able to interact with and bind to sample analytes. The microarrays are useful for screening large libraries of natural or synthetic compounds to identify natural binding partners for the binding elements, as well as to identify non-natural binding partners which may be of diagnostic or therapeutic interest. The invention is particularly useful in providing microarrays of antibodies or antibody fragments such as scFv, which have previously not been successfully incorporated into high-density arrays while maintaining their specific binding activity. The invention also provides methods for using such microarrays, methods for selecting epitopes for the antibodies or antibody fragments useful in such arrays, and methods for analyzing the data obtained from assays conducted on the microarrays.

Preferably, the immobilized binding elements are arranged in an array on a solid support, such as a silicon-based chip or glass slide. The surface of the support is chosen to possess, or are chemically derivatized to possess, at least one reactive chemical group that can be used for further attachment chemistry. There may be optional flexible molecular linkers interposed between the support and the binding elements. Examples of such linkers include bovine serum albumin (BSA) molecules, maleimide and vinyl sulfone groups.

In certain embodiments of the invention, a binding element is immobilized on a support in ways that separate the binding element's region responsible for binding to its cognate ligand and the region where it is linked to the support. In a preferred embodiment, the two regions are two separate termini, and the binding element is engineered to form covalent bond, through one of the termini, to a linker molecule on the support. Such covalent bond may be formed through a Schiff-base linkage, a linkage generated by a Michael addition, or a thioether linkage. In a particularly preferred embodiment, an antibody fragment is engineered to comprise a reduced cysteine at its carboxyl terminus.

In preferred embodiments, the microarrays comprise an array of immobilized yet functional binding elements at a density of at least 1000 spots per cm². In some embodiments, to prevent dehydration, the invention provides for adding a humectant such as glycerol to the layer of immobilized binding elements. In other embodiments, the invention provides for the addition of a blocking agent solution such as BSA to the substrate surface.

In another aspect, the present invention provides methods of labeling an antigen such that the labeling will not interfere with the antigen's binding with an antibody or antibody fragment. In a preferred embodiment, the antigen is labeled at its terminal amines after protease digestion. In a particularly preferred embodiment, the antigen is digested with trypsin before being labeled with a succinimidyl ester dye.

In a further aspect, the present invention provides a method for detecting a phorsphorylated protein by fragmenting a candidate protein into a plurality of peptides wherein one of the peptides comprises a known or suspected phorsphorylation site, and using an antibody or antibody fragment to select the peptide through an epitope close to the phorsphorylation site.

In yet another aspect, the present invention provides a method for identifying a small molecule that regulates protein-protein interaction. According to this aspect, a capture protein is attached to a support surface and exposed to its ligand and at least one small molecule. The presence or the absence of binding between the capture protein and the ligand is then detected to determine the regulatory effect of the small molecule. In a preferred embodiment, a microarray of capture proteins that act in the same cellular pathway are attached to the support surface to profile the regulatory effect of a small molecule on all these proteins in a parallel fashion.

In yet a further aspect, the present invention provides a method for studying a cellular event by attaching a capture molecule on a support surface to capture a cellular organelle contained in a solution such as a whole-cell lysate.

These and other aspects of the invention will be apparent to one of ordinary skill in the art from the following detailed disclosure, and description of the preferred embodiments.

### BRIEF DESCRIPTION OF THE DRAWINGS

FIG. 1A illustrates exemplary steps of treating a support surface to attach a BSA molecule to it and activating the BSA molecule.

FIG. 1B illustrates exemplary steps of attaching a capture protein to the activated BSA molecule.

FIG. 2 illustrates proximal phospho-affinity mapping.

FIGS. 3A and 3B illustrate an embodiment where small molecule regulating protein-protein interaction is studied.

FIG. 4A is a mass spectrometry profile of the steady state surface proteins from a trpsin digest of SKOV3 cells.

FIG. 4B is a mass spectrometry diagram showing peptide being affinity captured by scFv H7 on Ni-NTA SELDI surface.

FIG. 4C is a mass spectrometry diagram showing the result of a control experiment.

FIG. 4D illustrates the capture of transferrin receptor ectodomain tryptic peptide that is labeled with CY-5.

FIG. 5 are mass spectrometry diagrams showing binding by a fusion protein as a capture molecule versus the negative control.

FIG. 6 are mass spectrometry diagrams showing a small molecule competes a ligand off an binding elements on a SELDI surface.

FIGS. 7A and 7B show fluorescence units detected from ligand bound to immobilized binding elements in the presence or absence of a small molecule.

FIG. 8 shows fluorescence scans of microarrays that have captured labeled EGFR, TfR or ErbB2 at various dilutions.

FIG. 9 is a fluorescence scan showing labeled cell surface proteins from cell lysate being captured by antibody micoarrays.

FIG. 10 are fluorescence scans of microarrays where the capture of unlabeled antigen is detected through a second labeled antibody.

FIG. 11 are fluorescence scans detecting the binding of antigens from cell lysates. The detection is through a second labeled antibody.

### DETAILED DESCRIPTION OF THE INVENTION

The present invention depends, in part, upon the discovery of new methods of producing arrays, particularly microarrays, of naturally occurring or artificially produced biological macromolecules which may be used to screen samples, including both biological and artificial samples, to identify, isolate or quantify molecules in such samples that associate with the immobilized binding elements. Towards this end, the present invention provides methods and products to enable the high-throughput screening of very large numbers of compounds to identify those compounds capable of interacting with biological macromolecules.

The present invention has particularly significant applications in immunoassays, which pave the way for extensive and efficient screening using antibodies and similar molecules. Antibodies have long played an essential role in determining protein function, in identifying the spatiotemporal pattern of gene expression, in identifying protein-protein interactions, and for in vitro and in vivo target validation by phenotypic knockout. However, whereas individual antibodies are useful for monitoring individual proteins from biological samples, the present invention provides for the generation of large arrays of antibodies, antibody fragments, or antibody-like binding elements formatted for high throughput analysis. This technology, which enables comprehensive profiling of large numbers of proteins from normal and diseased-state serum, cells, and tissues, provides a powerful diagnostic and drug discovery tool.

One aspect of the present invention concerns improvements in methods of attaching a biomolecule to a solid support through a chemical linker, while retaining the biological functions of that molecule, particularly in the case of a capture protein or an antibody fragment.

I. Substrate/Support

The microarrays of the present invention are formed upon a substrate or support. Although the characteristics of these substrates may vary widely depending upon the intended use, the basic considerations regarding the shape, material and surface modification of the substrates are described below.

A. Shape

The substrates of the invention may be formed in essentially any shape. Although it is preferred that the substrate has at least one surface which is substantially planar or flat, it may also include indentations, protuberances, steps, ridges, terraces and the like. The substrate can be in the form of a sheet, a disc, a tubing, a cone, a sphere, a concave surface, a convex surface, a strand, a string, or a combination of any of these and other geometric forms. One can also combine several substrate surfaces to make use of the invention. One example would be to sandwich analyte-containing samples between two flat substrate surfaces with microarrays formed on both surfaces according to the invention.

B. Material

Various materials, organic or inorganic or a combination of both, can be used as support for this invention. Suitable substrate materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as substrate include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and various block co-polymers. The substrate can also comprise a combination of materials, whether water-permeable or not, in multi-layer configurations. A preferred embodiment of the substrate is a plain 2.5 cm×7.5 cm glass slide with surface Si—OH functionalities.

C. Surface Preparation/Reactive Groups

In order to allow attachment by a linker or directly by a binding element, the surface of the substrate may need to undergo initial preparation in order to create suitable reactive groups. Such reactive groups could include simple chemical moieties such as amino, hydroxyl, carboxyl, carboxylate, aldehyde, ester, ether (e.g. thio-ether), amide, amine, nitrile, vinyl, sulfide, sulfonyl, phosphoryl, or similarly chemically reactive groups. Alternatively, reactive groups may comprise more complex moieties that include, but are not limited to, maleimide, N-hydroxysuccinimide, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, fluorobenzene, biotin and avidin. Techniques of placing such reactive groups on a substrate by mechanical, physical, electrical or chemical means are well known in the art, such as described by U.S. Pat. No. 4,681,870, incorporated herein by reference.

To achieve high-density arrays, it may be necessary to “pack” the support surface with reactive groups to a higher density. One preferred method in the case of a glass surface is to first “strip” the surface with reagents such as a strong acid, and then to apply or reapply reactive groups to the surface.

In the case of a glass surface, the reactive groups can be silanes, Si—OH, silicon oxide, silicon nitride, primary amines or aldehyde groups. Slides treated with an aldehyde-containing silane reagent are preferred in immobilizing many binding elements and are commercially available from TeleChem International (Cupertino, Calif.) under the trade name “SuperAldehyde Substrates.” The aldehyde groups on the surface of these slides react readily with primary amines on proteins to form a Schiff base linkage. Since typical proteins display many lysine residues on their surfaces, as well as the generally more reactive α-amines at their N-termini, they can attach to the slide in a variety of orientations, permitting different sides of the protein to interact with other proteins or small molecules in solution. After arraying binding elements such as proteins onto these aldehyde slides, a buffer containing bovine serum albumin (BSA) may be applied to the slide to block later non-specific binding between analytes and unreacted aldehyde groups on the slide.

II. Linkers

Once the initial preparation of reactive groups on the substrate is completed (if necessary), linker molecules optionally may be added to the surface of the substrate to make it suitable for further attachment chemistry.

As used herein, the term “linker” means a chemical moiety which covalently joins the reactive groups already on the substrate and the binding element to be eventually immobilized, having a backbone of chemical bonds forming a continuous connection between the reactive groups on the substrate and the binding elements, and having a plurality of freely rotating bonds along that backbone. Linkers may be selected from any suitable class of compounds and may comprise polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. Preferably, the linker should be of an appropriate length that allows the binding element, which is to be attached, to interact freely with molecules in a sample solution and to form effective binding.

The linker in the present invention comprises at least two reactive groups with the first to bind the substrate and the second to bind the binding element. The two reactive groups may be of the same chemical moiety. The at least two reactive groups of linkers may include any of the chemical moieties described above of reactive groups on the substrate. And one preferred second group comprises a maleimide group. Another preferred embodiment for a linker's second group is a vinyl sulfone group. It is believed that the hydrophilicity of these groups helps limit nonspecific binding by analytes such as proteins when further assay is conducted in an aqueous buffer.

Methods for binding the linker to the surface of the substrate will vary depending on the reactive groups already on the substrate and the linker selected, and will vary as considered appropriate by one skilled in the art. For example, siloxane bonds may be formed via reactions between the trichlorosilyl or trisalkoxy groups of a linker and the hydroxyl groups on the support surface.

The linkers may be either branched or unbranched, but this and other structural attributes of the linker should not interfere stereochemically with relevant functions of the binding elements, such as a ligand-antiligand interaction.

Protection groups, known to those skilled in the art, may be used to prevent linker's end groups from undesired or premature reactions. For instance, U.S. Pat. No. 5,412,087, incorporated herein by reference, describes the use of photo-removable protection groups on a linker's thiol group.

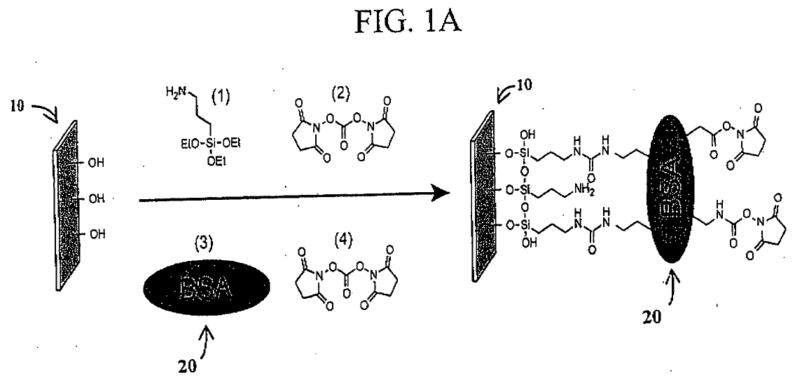

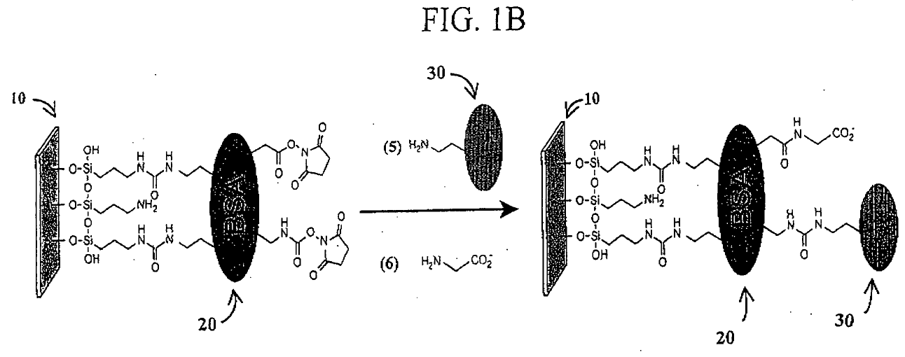

In a preferred embodiment, the linker comprises a BSA molecule. An example of such an embodiment is a BSA-NHS slide suitable for making microarrays. Although appropriate for some applications, slides functionalized with aldehyde groups, further blocked with BSA, are not suitable when peptides or small proteins are arrayed, presumably because the BSA obscures the molecules of interest. For such applications, BSA-NHS slides are preferred. FIGS. 1A and 1B illustrate a method of making such a slide. First, a molecular monolayer of BSA is attached to the surface of a glass slide. Specifically shown in FIG. 1A, a glass slide 10 with hydroxyl groups is silanated with aminopropyl triethoxy silane (step 1) before being activated with N,N′-disuccinimidyl carbonate (step 2). The activated amino group on the slide in turn forms covalent bonds with linker 20, which is BSA (step 3). Then, the surface of the BSA is activated with N,N′-disuccinimidyl carbonate (step 4), resulting in activated carbamate and ester, such as a N-hydroxy succinimide (NHS) group. Referring to FIG. 1B, the activated lysine, aspartate, and glutamate residues on the BSA react readily with the surface amines on the binding element 30, which is a capture protein here (step 5) to form covalent urea or amide linkages. Any remaining reactive groups on BSA are subsequently quenched with glycine (step 6). The result is a binding element 30 (a capture protein here) immobilized to a support 10 through a linker 20 (a BSA molecule here). In contrast to the BSA-blocked slides with aldehyde functionality, proteins or peptides arrayed on BSA-NHS substrates are displayed on top of the BSA monolayer, rendering them accessible to macromolecules in solution.

III. Binding Elements

The binding elements of the present invention may be chosen from any of a variety of different types of naturally occurring or synthetic molecules, including those having biological significance (“biomolecules”).

For example, the binding elements may include naturally occurring molecules or molecule fragments such as nucleic acids, nucleic acid analogs (e.g., peptide nucleic acid), polysaccharides, phospholipids, capture proteins including glycoproteins, peptides, enzymes, cellular receptors, and immunoglobulins (e.g., antibodies, antibody fragments,) antigens, naturally occurring ligands, other polymers, and combinations of any of the above. And it is also contemplated that natural product-like compounds, generated by standard chemical synthesis or from split-and-pool library or parallel syntheses, may be utilized as binding elements.

A. Antibodies and Antibody Fragments

Antibodies and antibody fragments are preferred candidates for binding elements. These include antigen-binding fragments (Fabs), Fab′ fragments, pepsin fragments (F(ab′)₂ fragments), scFv, Fv fragments, single-domain antibodies, dsFvs, Fd fragments, and diabodies, as well as full-length polyclonal or monoclonal antibodies. Antibody-like fragments, such as modified fibronectin, CTL-A4, and T cell receptors are contemplated here as well. Once the microarray has been formed, the antigen binding domains of the antibodies or antibody fragments may be utilized to screen for molecules with the specific antigenic determinants recognized by the antibodies or antibody fragments.

In a preferred embodiment, to study cellular translocation events and cell surface expression, phage-displayed scFv that trigger cell internalization of a surface receptor can be directly selected from large non-immune phage libraries by recovering and amplifying phage particles from within the cells. See Becerril et al. (1999), Biochem Biophys Res Commun. 255(2): 386-93, the entire disclosure of which is incorporated by reference herein.

B. Receptors

Naturally occurring biological receptors, or synthetically or recombinantly modified variants of such receptors, also may be used as the binding elements of the invention. Classes of receptors that can be used as binding elements include extracellular matrix receptors, cell-surface receptors and intracellular receptors. Specific examples of receptors include fibronectin receptors, fibrinogen receptors, mannose 6-phosphate receptors, erb-B2 receptors, and EGF (epidermal growth factor) receptors.

C. Receptor Ligands

Similarly, naturally occurring biological receptor ligands, or synthetically or recombinantly modified variants of such ligands, also may be used as binding elements to screen for their specific binding partners, or for other, non-natural binding partners. Classes of such ligands include hormones, growth factors, neurotransmitters, antigens and can be phage-displayed.

D. Modifications for Coupling to Substrate/Linkers

As will be apparent to those of skill in the art, the binding elements may be modified in order to facilitate attachment, through covalent or non-covalent bonds, to the reactive groups on the surface of the substrate, or to the second reactive groups of a linker attached to the substrate. As examples of such modifications, nucleophilic S-, N- and O-containing groups may be added to facilitate attachment of the binding element to the solid support via a Michael addition reaction to the linker.

To preserve the binding affinity of an binding element, it is preferred that the binding element is modified so that it binds to the support substrate at a region separate from the region responsible for interacting with the binding element's cognate ligand. If the binding element binds its ligand at a first terminus, attaching the binding element to the support at a second or opposite terminus, or somewhere in between the termini may be such a solution. In a preferred embodiment, where the binding element is an scFv, the present invention provides a modification method such that the scFv can be attached to the surface of a glass slide through binding with an electrophilic linker, such as a maleimide group, without interfering with the scFv's antigen-binding activity. According to this method which is detailed in Example C (i), an scFv is first engineered so that its carboxy-terminus includes a cysteine residue which can then form a covalent bond with an electrophilic linker such as the maleimide group. Similarly, a binding element's N-terminus can be engineerd to include a reactive group for attachment to the support surface.

E. Coupling to Substrates/Linkers

Methods of coupling the binding element to the reactive end groups on the surface of the substrate or on the linker include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the substrate/linker and binding element.

As discussed in the Examples section below, a Michael addition may be employed to attach compounds to glass slides, and plain glass slides may be derivatized to give surfaces that are densely functionalized with maleimide groups. Compounds containing thiol groups, such as an scFv modified to include a cysteine at the carboxy-terminus, may then be reacted with the maleimides to form a thioether linkage.

IV. Formation of Microarrays

In one aspect, the present invention provides methods for the generation of arrays, including high-density microarrays, of binding elements immobilized on a substrate directly or via a linker. According to the methods of the present invention, extremely high density microarrays, with a density over 100, preferably over 1000, and further preferably over 2000 spots per cm², can be formed by attaching a biomolecule onto a support surface which has been functionalized to create a high density of reactive groups or which has been functionalized by the addition of a high density of linkers bearing reactive groups.

A. Spotting

The microarrays of the invention may be produced by a number of means, including “spotting” wherein small amounts of the reactants are dispensed to particular positions on the surface of the substrate. Methods for spotting include, but are not limited to, microfluidics printing, microstamping (see, e.g., U.S. Pat. No. 5,515,131 and U.S. Pat. No. 5,731,152), microcontact printing (see, e.g., PCT Publication WO 96/29629) and inkjet head printing. Generally, the dispensing device includes calibrating means for controlling the amount of sample deposition, and may also include a structure for moving and positioning the sample in relation to the support surface.

(i) Volume/Spot Size

The volume of fluid to be dispensed per binding element in an array varies with the intended use of the array, and available equipment. Preferably, a volume formed by one dispensation is less than 100 nL, more preferably less than 10 nL, and most preferably about 1 nL. The size of the resultant spots will vary as well, and in preferred embodiments these spots are less than 20,000 μm in diameter, more preferably less than 2,000 μm in diameter, and most preferably about 150-200 μm in diameter (to yield about 1600 spots per square centimeter).

(ii) Viscosity Additives

The size of a spot in an array corresponding to a single binding element spot may be reduced through the addition of media such as glycerol or trehalose that increase the viscosity of the solution, and thereby inhibit the spreading of the solution. Hydrophobic boundaries on a hydrophilic substrate surface can also serve to limit the size of the spots comprising an array.

Adding a humectant to the solution of the binding element may also effectively prevent the dehydration of the microarrays, once they are created on the surface of the substrate. Because dehydration can result in chemical or stereochemical changes to binding elements, such as oxidation or, in the case of proteins, denaturation, the addition of a humectant can act to preserve and stabilize the microarray and maintain the functionality of binding elements such as scFv. For example, in some preferred embodiments, scFv are coupled to maleimide-derivatized glass in phosphate-buffered saline (PBS) solutions with 40% glycerol. The glycerol helps maintain continued hydration which, in turn, helps to prevent denaturation.

(iii) Blocking Agents

Solutions of blocking agents may be applied to the microarrays to prevent non-specific binding by reactive groups that have not bound to a binding element. Solutions of bovine serum albumin (BSA), casein, or nonfat milk, for example, may be used as blocking agents to reduce background binding in subsequent assays.

(iv) Robotics

In preferred embodiments, high-precision, contact-printing robots are used to pick up small volumes of dissolved binding elements from the wells of a microtiter plate and to repetitively deliver approximately 1 nL of the solutions to defined locations on the surfaces of substrates, such as chemically-derivatized glass microscope slides. Examples of such robots include the GMS 417 Arrayer, commercially available from Affymetrix of Santa Clara, Calif., and a split pin arrayer constructed according to instructions downloadable from http://cmgm.stanford.edu/pbrown. The chemically-derivatized glass microscope slides are preferably prepared using custom slide-sized reaction vessels that enable the uniform application of solution to one face of the slide as shown and discussed in the Examples section. This results in the formation of microscopic spots of compounds on the slides. It will be appreciated by one of ordinary skill in the art, however, that the current invention is not limited to the delivery of 1 nL volumes of solution, to the use of particular robotic devices, or to the use of chemically derivatized glass slides, and that alternative means of delivery can be used that are capable of delivering picoliter or smaller volumes. Hence, in addition to a high precision array robot, other means for delivering the compounds can be used, including, but not limited to, ink jet printers, piezoelectric printers, and small volume pipetting robots.

B. In Situ Photochemistry

In forming arrays or microarrays of molecules on the surface of a substrate, in situ photochemistry maybe used in combination with photoactivatable reactive groups, which may be present on the surface of the substrate, on linkers, or on binding elements. Such photoactivatable groups are well known in the art.

C. Labeling

Binding elements may be tagged with fluorescent, radioactive, chromatic and other physical or chemical labels or epitopes. For certain preferred embodiments where quantified labeling is possible, this yields great advantage for later assays.

In a preferred embodiment, a fluorescent dye containing a hydrophilic polymer moiety such as polyethyleneglycol is used.

V. Samples for Assays

Upon formation of microarrays of binding elements on the solid support, large quantities of samples may be applied to the support surface for binding assays. Examples of such samples are as follows:

A. Body Fluids/Tissue and Biopsy Samples

Samples to be assayed using the microarrays of the present invention may be drawn from various physiological, environmental or artificial sources. In particular, physiological samples such as body fluids of a patient or an organism may be used as assay samples. Such fluids include, but are not limited to, saliva, mucous, sweat, whole blood, serum, urine, genital fluids, fecal material, marrow, plasma, spinal fluid, pericardial fluids, gastric fluids, abdominal fluids, peritoneal fluids, pleural fluids and extraction from other body parts, and secretion from other glands. Alternatively, biological samples drawn from cells grown in culture may be employed. Such samples include supernatants, whole cell lysates, or cell fractions obtained by lysis and fractionation of cellular material.

B. Cell Extracts

Extracts of cells and fractions thereof, including those directly from a biological entity and those grown in an artificial environment, can also be used to screen for molecules in the lysates that bind to a particular binding element.

C. Normal v. Diseased Samples

Any of the above-described samples may be derived from cell populations from a normal or diseased biological entity.

D. Treated v. Untreated Samples

Any of the above-described samples may be derived from cell populations which have or have not been treated with compounds or other treatments which are believed or suspected of being either deleterious or beneficial, and differences between the treated and untreated populations may be used to assess the effects of the treatment.

E. Labeling

Specific molecules in a given sample may be modified to enable later detection by using techniques known to one of ordinary skill in the art, such as using fluorescent, radioactive, chromatic and other physical or chemical labels. In a preferred embodiment, a fluorescent dye containing a hydrophilic polymer moiety such as polyethyleneglycol (e.g. fluorescin-PEG2000-NHS) is used. Labeling can be accomplished through direct labeling of analytes in the sample, or through labeling of an affinity tag that recognizes an analyte (indirect labeling). Direct labeling of sample analytes with different fluorescent dyes makes it possible to conduct multiple assays from the same spot (e.g., measuring target protein's expression level and phosphorylation level). When the analyte is a phage-displayed ligand, the phage may be pre-labeled for detecting binding between the ligand and the microarray of binding elements.

Under the direct-labeling approach, sample over-labeling has long been recognized as a serious problem. Over-labeling of proteins can cause aggregation of protein conjugate, which tends to result in non-specific staining; it can also reduce antibody's specificity for its antigen by disrupting antibody's epitope-recognition function, causing loss of signal. It is well known in the art that, to mitigate over-labeling, one need to either shorten reaction time for the labeling process or increase substrate:label ratio. A solution to over-labeling is to first digest a whole protein into peptides and then label the termini of the peptides, which avoids labeling any internal epitopes. Accordingly, the labeling process may proceed to completion without one having to worry about over-labeling and thus giving a researcher more complete control over the labeling process. Moreover, if the potential labeling sites on a peptide is known, it is possible to quantify labeled peptide once the peptide is captured through affinity reagents that recognize an internal epitope. An application of this method would be to quantify labeled peptides digested from whole proteins in cell extracts for quantitative analysis of protein expression levels.

In a preferred embodiment, whole proteins are digested with trypsin before subjected to labeling by a succinimidyl ester dye such as Cy3, Cy5 or an Alexa dye. A succinimidyle ester dye labels primary amines, such as the one in lysine. Trypsin cleaves after lysines and generates peptides with lysines at their C-terminus. Therefore, peptides resulting from trypsin digestion fall into two categories: those without lysine and having a primary amine at the N-terminus, and those with a lysine at the C-terminus and hence primary amines at both termini. None of the peptide would have any internal lysine. As a result, a succinimidyl ester dye will only label tryptic peptides at their termini without labeling any internal epitope.

In an alternative embodiment, one may use a protease other than trypsin to digest a whole protein and still use a succinimidyl ester dye for labeling as long as the peptide to be captured does not contain an internal lysine. That way, labeling will still only occur at a terminus of the selected peptide. Such a peptide may be used as a preferential panning peptide. To take advantage of a preferential panning peptide, an immunoglobulin is first raised against the peptide. Second, a sample, e.g., from a whole cell lysate, is digested with a protease or a combination of proteases that will generate that specific panning peptide, resulting in a library of peptides. These peptides are then labeled to completion with a succinimidyl ester dye. A large excess of reactive labeling reagent may be used to ensure complete labeling of the non-lysine containing peptide. Then, the labeled peptides are applied to the immunoglobulin for capture.

Because the amount of labeling on a preferential panning peptide is known, one can quantify the amount of such peptide in a given sample through the amount of label signals detected after affinity capture. Once the number of such panning peptides resulting from the protease digestion of one target protein is known, that number can be easily translated into the amount of the target protein in the sample. Amino acids other than lysine can also be targeted for use with this method. For example, proteins with limited number of natural or added cysteine may be selected or constructed to be labeled, via a reduced thiol with maleimide-coupled dye such as maleimide-coupled Alexa 488 (commercially available from Molecular Probes of Eugene, Oreg.).

Indirect labeling of an antigen analyte may be achieved by using a second antibody or antibody fragment that has been labeled for subsequent detection (e.g., with radioactive atoms, fluorescent molecules) in a sandwiched fashion. In a preferred embodiment, an antigen that binds to a microarray of antibodies is detected through a second fluorescently labeled antibody to the antigen, obviating the need for labeling the antigen. In a further preferred embodiment, the second antibody is a labeled phage particle that displays an antibody fragment. Standard phage display technology using phages such as M13 may be used to produce phage antibodies including antibody fragments such as scFv. This allows relatively easy and fast production of reagents for sandwich detection from phage display antibody libraries. To ensure that the phage antibodies recognize an epitope different from the one that the immobilized capture antibody recognizes on the antigen, selection from phage display libraries may be carried out in the following way: (1) tubes are coated with the same antibody that is immobilized in microarray for capture purpose, (2) the tube is blocked and the antigen is added and captured by the coated antibody, (3) after washing, phage antibody libraries may be panned in the tubes. The isolated phage antibodies (or polyclonal phage antibody) will only bind epitopes distinct from the epitope the capture antibody recognizes, and are thus ideal for the sandwich detection approach.

F. Contact Time

Binding assays can be performed by exposing samples to the surface prepared according to methods described above. Such a surface is first exposed to a sample solution and then incubated for a period of time appropriate for each specific assay, which largely depends on the time needed for the expected binding reactions. This process can be repeated to apply multiple samples either simultaneously or sequentially. Sequential application of multiple samples generally requires washes in between.

VI. Binding Assays

A surface prepared according to the methods described above can be used to screen for molecules in a sample that have high affinity for the binding elements attached to the surface. Specific binding may be detected and measured in a number of different ways, depending on the way the target molecules in the sample are labeled, if at all. A common example is to use the technique of autoradiography to detect binding of molecules pre-labeled with radioactive isotopes.

In a preferred embodiment, fluorescent dyes (CY5) were used to label proteins in a given sample before the sample was applied to a slide surface printed with microarrays of functional scFv. After incubation and washes, the slide surface was then dried and imaged on a molecular dynamics STORM or ArrayWorx™ optical reader from Applied Precision of Seattle, Wash.

In another preferred embodiment, secondary antibodies labeled with fluorochromes such as CY3 were used for later detection of a primary antibody participating in the binding.

Various detection methods known in the art such as mass spectrometry, surface plasmon resonance, and optical spectroscopy, to name a few, can be used in this invention to allow detection of binding even if binding targets are not labeled at all.

VII. Analysis of Assay Results

A. Detecting Presence/Absence in Samples

This invention can be used to confirm the presence or the absence, in a biological sample, of a binding partner to a molecule of interest.

B. Determining Ratios Between Samples

Ratios of gene and protein expression in different cell populations, such as between a normal and a diseased state, can be calculated for comparison.

VIII. Applications/Utilities

Because the molecules of biological significance that can be studied by this invention include, but are not limited to, those involved in signal transduction, apoptosis, dimerization, gene regulation, cell cycle and cell cycle checkpoints, and DNA damage checkpoints, the present invention has broad applications in the research of biological sciences and medicine.

As will also be appreciated by one of ordinary skill in the art, protein arrays may also be useful in detecting interactions between the proteins and alternate classes of molecules other than biological macromolecules. For example, the arrays of the present invention may also be useful in the fields of catalysis, materials research, information storage, separation sciences, to name a few.

A. Target Discovery

It will be appreciated by one of ordinary skill in the art that the generation of arrays of proteins having extremely high spatial densities facilitates the detection of binding and/or activation events occurring between proteins of a defined set and biological macromolecules. Thus, the present invention provides, in one aspect, a method for identifying molecular partners and discovering binding targets for macromolecules of biological significance. The partners may be proteins that bind to particular macromolecules of interest and are capable of activating or inhibiting the biological macromolecules of interest. In general, this method involves (1) providing an array of one or more proteins, as described above, wherein the array of proteins has a density of at least 1,000 spots per cm² (2) contacting the array with one or more types of biological macromolecules of interest; and (3) determining the interaction between specific proteins and macromolecule partners.

In a particularly preferred embodiment the inventive arrays are utilized to identify compounds for chemical genetic research. In classical genetics, either inactivating (e.g., deletion or “knock-out”) or activating (e.g., oncogenic) mutations in DNA sequences are used to study the function of the proteins that are encoded by these genes. Chemical genetics instead involves the use of small molecules that alter the function of proteins to which they bind, thus either inactivating or activating protein function. This, or course, is the basis of action of most currently approved small molecule drugs. The present invention involved the development of “chip-like” technology to enable the rapid detection of interactions between small molecules and specific proteins of interest. The methods and composition of the present invention can be used to identify small molecule ligands for use in chemical genetic research. One of ordinary skill in the art will realize that the inventive compositions and methods can be utilized for other purposes that require a high density protein format.

B. Signal Transduction

Another preferred embodiment of the binding assays performed in this invention is to study modulation of protein-protein interaction by small molecules. These assays measure either the facilitation or competition for cognate binding by different molecules in order to help understand aspects of binding dynamics under varying conditions. In an exemplary embodiment, a capture protein is attached on a support surface in microarray, cognate ligands are added to bind to the capture protein. The binding between the capture protein and its cognate ligand is monitored and compared in the presence or absence of a small molecule that may be a drug candidate. In a preferred embodiment, various capture proteins's interaction with various ligands affected by various small molecules are investigated in a multi-plex fashion on a microarray chip.

Protein interactions often occur through domains that are sometimes called binding motifs. It is in these regions that small molecules that are effective at regulating protein interactions are most likely to work. However, proteins within a family tend to share homologous sequences that contribute to forming binding motifs and proteins that contain these motifs often have similar functions. A problem in screening for drugs that regulate such protein functions is obtaining specificity in these screens as the targets among the binding motif family of proteins are similar in structure, and have similar binding features. The protein microarray technology disclosed here permits efficient and easily repeatable steps for determine specificity of small molecules for regulating large numbers of motif-containing protein family members, and will greatly facilitate the process of drug screening.

In an exemplary embodiment, regulation of the Bcl-2 family, known to affect cell apoptosis, is studied. These proteins share homology to combinations of four Bcl-2 homology regions (BH1-4). The Bcl-2 family proteins function to either protect cells against apoptosis or to promote apoptosis by regulating membrane behavior and ion channel function at the mitochondria and the endoplasmic reticulum. The anti-apoptotic family members, Bcl-2, Bcl-XL, and Mcl-1 contain all four domains. The largest group of pro-apoptotic members, Bad, Bik, Bid, Bag-1, HRK, and Noxa contain only BH-3 domains, while pro-apoptotic proteins Bax and (Multidomain pro-apoptotic proteins) contain BH-1, BH-2, and BH-3 domains.

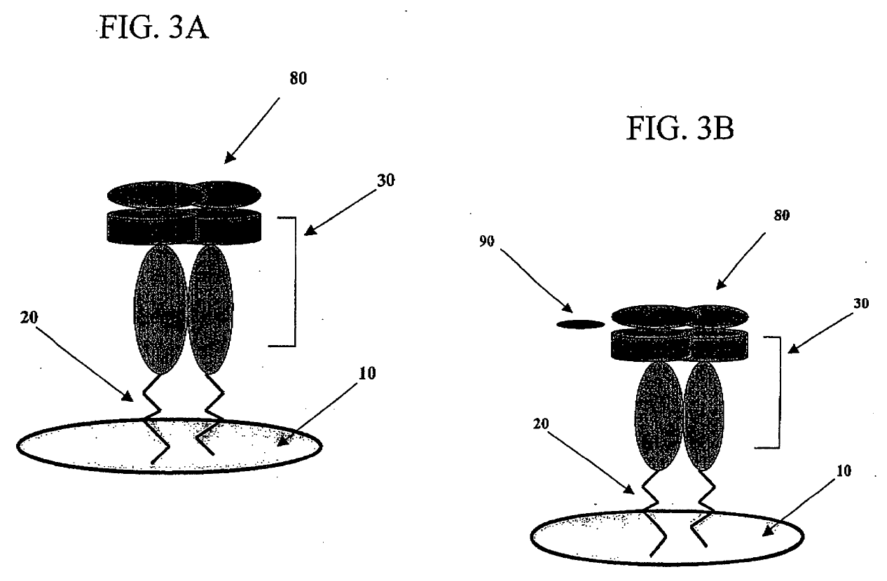

Methods of the invention can be used to screen for small molecules that regulate the function of an entire family of apoptosis-regulating proteins. Such a small molecule may mimic the function of a BH-3 protein and serve as a drug candidate. Referring to FIGS. 3A and 3B, recombinant fusion proteins from the Bcl-2 family of apoptosis regulating proteins may be prepared by standard methods and printed in microarrays as binding element 30 on either BSA-NHS glass slides or an aldehyde derivatised glass slide 10 as described earlier through a linker 20. Ligands 80 for these proteins such as a full length Bcl-XL protein may be added in the absence or presence of a small molecule 90 such as a BH-3 containing peptide from the Bcl-2 family protein BAK or a small molecule that mimics a BH-3 containing peptide. The ligand 80 may be labeled with a fluorescent dye (e.g. CY5). Concentration of the printed proteins, the ligands, or the small molecule may be varied, by itself or in combinations with others. The slides may then be read using an optical reader such as the Arrayworx scanner and/or confirmed through mass spectrometry using commerically available mass spectrometry chips. The increase or decrease in the signal obtained from bound ligand can be used to chart the regulatory roles of the small molecule, whether it is up-regulatory or down-regulatory. Using the method of the invention, multiple capture molecules, multiple ligands and multiple small molecules can be screened side by side on a single array support (e.g. a 96 well plate), greatly increasing efficiency in drug screening. A more detailed example can be found in the Example Section E (iii).

Another example of the invention's application in studying signal transduction is to screen for small molecules that inhibit protein-protein binding in the apoptotic pathway through the BH-4 region of multidomain-containing BCl-2 family members.

C. Protein Expression

To date, there are no published reports on microarray-based detection of proteins in labeled cell extracts. Labeling and detection of cell surface proteins would allow parallel profiling of multiple cell surface antigens. State of the art in cell surface molecule profiling is by flow cytometry or fluorescence microscopy, currently allowing 2-5 different antigens to be profiled in a single sample. Antibody arrays in theory allow the detection of an unlimited number of antigens. Furthermore, antibody arrays have the potential for detecting intracellular proteins and protein modifications such as phosphorylation in parallel with expression.

In an exemplary embodiment, monoclonal antibodies to cell surface proteins such as c-ErbB2, EGFR, and transferrin receptor are arrayed on a BSA-NHS slide by a GMS 417 arrayer. Live cells from a cancerous cell line such as the epidermoid carcinoma cell line A-431 or breast cancer cell line SK-BR-3 may be used as sample cells. Cell surface proteins are preferably labeled with a dye that contains a hydrophilic polymer moiety such as a polyethyleneglycol, which has shown good specificity, low background, and does not label proteins inside cells. An example of such a dye is fluorescein-PEG2000-NHS dye available from Shearwater. Following labeling and wash, cells are lysed (e.g., in SDS). Total labeled proteins are then incubated on the antibody microarray for binding to occur before the slides are scanned by an optical reader. As a result, it was confirmed that the A-431 cell line over-expresses EGFR but not ErbB2. Likewise, it was confirmed that the SK-BR-3 cell line over-expresses ErbB2, but not EGFR.

D. Post-Translational Modification

Protein function is often regulated by post-translational modifications such as the addition of sugar complexes, lipid anchors such as provided by myristoylation, geranyl-geranylation or farnesylation, or by phosphorylation to mention a few. The regulation of protein function by phoshorylation or dephosphorylation is central in cell signal transduction.

Methods of the present invention can be used to study post-translational events or to identify phosphorylation sites. In a preferred embodiment, antibody fragments such as scFv are printed on Matrix-Assisted Laser Desportion/Ionization (MALDI) chips for detecting phosphorylation of known and suspected phosphorylation sites in proteins. Coupling proteins to reactive surface MALDI mass spectrometry surfaces was described in U.S. Pat. No. 6,020,208, and incorporated herein by reference. The chip is commercially available from Ciphergen Biosystems Freemont, Calif. In an exemplary embodiment, phosphospecific antibodies against the apoptotic proteins Bcl-2, Bad, and caspase 9 are coupled to reactive surface MALDI chips, and are used for selective capture of phosphorylated fragments of these proteins. The chip can be analyzed for mass using time of flight mass spectrometry.

Methods of the present invention further provide a new way to detect the occurrence of a phosphorylation event on a known or unknown phospho-accepting residue using recombinant single chain antibodies (scFv) coupled with mass spectrometry. This method has been termed proximal phospho-affinity mapping, and serves as an alternative method that does not rely on the use of IMAC or the use of phospho-specific antibodies, which are notoriously difficult to make.

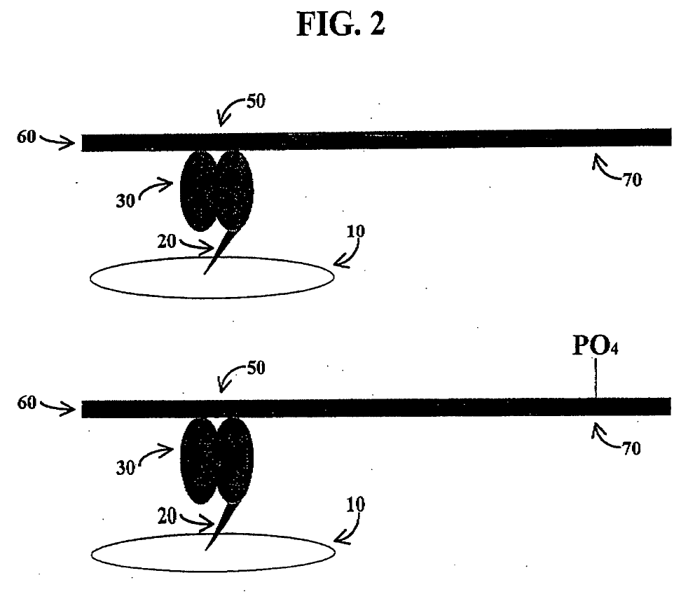

Referring to FIG. 2, an embodiment of this method uses recombinant single chain antibodies (scFv), polyclonal, or monoclonal antibodies 30 that are designed to recognize, instead of a phorsphorylation site 70 itself, an epitope 50 on the same antigen that is in proximity to the phosphorylation site 70, whether site 70 is confirmed or just suspected for phosphorylation. The epitope 50 may be as close as 5-10 amino acids away, as long as the distance between the epitope 50 and the phosphorylation site 70 is such that antibody recognition is not hindered by a phorsphorylation event. Such an antibody or antibody fragment 30, which is coupled to a support surface 10 through a linker 20, will recognize the antigen 60 (e.g. a tryptic peptide) whether or not the antigen is phosphorylated. In an exemplary embodiment, peptides are generated using proteases such as trypsin or V8, or by non-enzymatic methods, such as CNBr. This yields peptide fragments that can be identified by their unique sizes. Among these fragments are the target fragments 60 that contains known or predicted phosphorylation sites. Single chain antibodies or traditional antibodies are panned or immunized against synthetic peptides that correspond to an epitope region 50 that is close to the phosphorylation site 70 in the tryptic fragment 60 using standard panning procedures. The epitope 50 may consist of as few as 3-7 amino acids. The antibody or antibody fragment that are generated may be used as capture molecule coupled to MALDI reactive chips. The chips may then be used to detect characteristic mass shift indicative of phosphorylation. Since this method enables parallel purification/identification and analysis of phosphorylation, it offers a valuable detection tool for phosphorylation screening. And because the antibody or antibody fragment generated according to this method recognizes the target peptide in both the phosphorylated and unphosphorylated state, this method is also useful in studying events and conditions that affect phosphorylation.

In a particularly preferred embodiment, the peptide 60 is selected in the following way: first, kinase substrate consensus sequences are located in the target protein through searches conducted in a database that contains protein sequence information. Then, a peptide containing such consensus sequence is selected through comparing the digestion maps of various proteases—peptides of about 20 amino acids are preferred. Last, an epitope other than the kinase substrate consensus sequences on the selected peptide is chosen for raising an antibody or antibody fragment.

E. Cellular Organelle

Methods of the invention can also be used to capture cellular organelles organelles' from whole cell extracts or from fractions of whole cell extracts. In a preferred embodiment, an antibody that recognizes a voltage dependent anion channel (“VDAC”) receptor uniquely associated with the mitochondrial membrane is printed as described earlier to capture Green Fluorescent-coupled cytochrome C expressing mitochondria. Dyes that have potentiometric quality can be used to specifically label mitochondria that have intact voltage gradient. The detection of captured mitochondria or other organelles from cells at different states can be used to indicate occurrence of apoptosis or other cellular events.

F. Others

Methods of the invention may also be used for other applications such as tissue typing, disease diagnosis, and evaluation of therapeutics. Biological samples from patients that may reveal genetic disorders (PCT patent publication No. 89/11548, incorporated herein by reference), may be used in the present invention. Likewise, this invention can be used to detect abnormality in protein expressions, the existence of antigens or toxins in a given sample. Further, methods of the invention can also be used to evaluate responses from organisms, tissues or individual cells to exposure to drugs, pharmaceutical lead compounds, or changes in environmental factors.

### EXAMPLES

A. Substrate Surface Preparation

(i) Method of Stripping Glass Slide and Re-Packing with Reactive Groups

An example of this preferred method is as follows: first, a plain glass slide (VWR Scientific Products, for instance) is cleaned in a piranha solution (70:30 v/v mixture of concentrated H₂SO₄ and 30% H₂O₂) for 12 hours at room temperature. (Caution: “piranha” solution reacts violently with several organic materials and should be handled with extreme care). After thorough rinsing with water, the slides is treated with a silane solution, such as a 3% solution of 3-aminopropyltriethoxysilane in 95% ethanol. And before treating the slides, the silane solution may be stirred for at least 10 minutes to allow hydrolysis and silanol formation. The slide is then briefly dipped in ethanol or like solutions and centrifuged to remove excess silanol. The adsorbed silane layer is then cured (e.g., one hour at 115° C.). After cooling, the slide is washed in ethanol or like solutions to remove uncoupled reagent.

A simple, semi-quantitative method can be used to verify the presence of amino groups on the slide surface. An amino-derivatized slide is washed briefly with 5 mL of 50 mM sodium bicarbonate, pH 8.5. The slide can then be dipped in 5 mL of 50 mM sodium bicarbonate, pH 8.5 containing 0.1 mM sulfo-succinimidyl-4-0-(4,4′-dimethoxytrityl)-butyrate (s-SDTB; Pierce, Rockford, Ill.) and shaken vigorously for 30 minutes. (The s-SDTB solution may be prepared by dissolving 3.03 mg of a s-SDTB in 1 mL of DMF and diluting to 50 mL with 50 mM sodium bicarbonate, pH 8.5). After a 30-minute incubation, the slide can then be washed with 20 mL of distilled water and subsequently treated with 5 mL of 30% perchloric acid. The development of an orange-colored solution will indicate that the slide has been successfully derivatized with amines; no color change has been seen for untreated glass slides. Quantitation of the 4,4′-dimethoxytrityl cation (E₄₉₈ₙₘ=70,000 M⁻¹cm⁻¹) released by the acid treatment has indicated an approximate density of 2 amino groups 2 per nm².

B. Addition of Linkers to Substrates

(i) BSA as Linker

BSA-NHS slides, displaying activated amino and carboxyl groups on the surface of an immobilized layer of bovine serum albumin (BSA), were fabricated as follows: 10.24 g N,N′-disuccinimidyl carbonate (100 mM) and 6.96 ml N,N-diisopropylethylamine (100 mM) were dissolved in 400 ml anhydrous N,N-dimethylformamide (DMF). Thirty polylysine slides, such as CMT-GAP slides (Corning Incorporated, Corning, N.Y.), displaying amino groups on their surface, were immersed in this solution for 3 hr at room temperature. These slides were rinsed twice with 95% ethanol and then immersed in 400 ml of phosphate buffered saline (PBS), pH 7.5 containing 1% BSA (w/v) for 12 hr at room temperature. Slides were further rinsed twice with ddH₂O, twice with 95% ethanol, and centrifuged at 200 g for 1 min to remove excess solvent. Slides were then immersed in 400 ml DMF containing 100 mM N,N′-disuccinimidyl carbonate and 100 mM N,N-diisopropylethylamine for 3 hr at room temperature. Slides were rinsed four times with 95% ethanol and centrifuged as above to yield BSA-NHS slides. Slides were stored in a desiccator under vacuum at room temperature for up to two months without noticeable loss of activity.

(ii) A Malemide Group as Linker

Maleimide-derivatised slides were manufactured as follows: after the surface of a plain glass' slide was “packed” (re-silanated, for instance) as described in the Example A(i), the resulting slides were transferred to slide-sized polydimethylsiloxane (PDMS) reaction vessels. One face of each slide was treated with 20 mM N-succinimidyl 3-maleimido propionate in 50 mM sodium bicarbonate buffer, pH 8.5, for three hours. (This solution was prepared by dissolving the N-succinimidyl 3-maleimido propionate in DMF and then diluting 10-fold with buffer). After incubation, the plates were washed several times with distilled water, dried by centrifugation, and stored at room temperature under vacuum until further use. The resulting slide surface was equipped with a maleimide end.

C. Preparation of Binding Elements

(i) Production and Purification of Cysteine-Tagged scFv

The scFv C6.5 binds to the extracellular region of the human tumor antigen c-erbB-2 with a Kd of 1.6×10⁻¹⁰ M. This antibody was isolated using affinity driven selection as described in Schier et al. (1996), J. Mol. Biol. 255(1):28-43.

The gene for the scFv C6.5 was then subcloned into a pUC-119-(Hexa-His)-Cys expression vector, which results in the addition of a hexa-His tag followed by a single cysteine to the COOH-terminus of the scFv. The protein was expressed and purified using immobilized metal affinity chromatography (IMAC). Binding affinity mutants of C6.5 were made by mutagenizing the complementary binding region (CDR), and the affinity constants of the derivative mutants [C6.5ML 3-4(Kd=3.4×10⁻⁹) and C6.5G98 (Kd=1.6×10⁻⁹)], were determined using BiaCore (described in Schier et al 1996b). The cysteine tagged scFv C6.5, C6.5ML3-4, and C6.5 G98. were used to demonstrate ligand capture by scFv which have been chemically coupled to glass surfaces. The reduced sulfhydryl of the COOH terminal cysteine of these scFv yields a thiol that can be used to couple the scFv to glass surfaces that have been functionalized with maleimide groups.

(ii) Reducing an Scfv for Conjugation to a Maleimide Linker

Purified scFv were reduced with 5 mM cysteamine (SIGMA) for 1 hour at 25° C. and exchanged into phosphate buffered saline(PBS), pH7.0 using a P10 spin column.

D. Assays Employing Microarrays

(i) Scanning Slides for Fluorescence

Slides were scanned using an Array WoRox™ slide scanner (AppliedPrecision, Issaquah, Wash.). Slides were scanned at a resolution of 5 μm per pixel. Double filters were employed for both the incident and emitted light. Fluorescein fluorescence was observed using a FITC/FITC excitation/emission filter set, Cy3 fluorescence was observed using a Cy3/Cy3 excitation/emission filter set, and Cy5 fluorescence was observed using a Cy5/Cy5 excitation/emission filter set.

E. Applications of Microarrays (i) Affinity Capture of Labeled Peptides on scFv Modified Glass Surfaces.

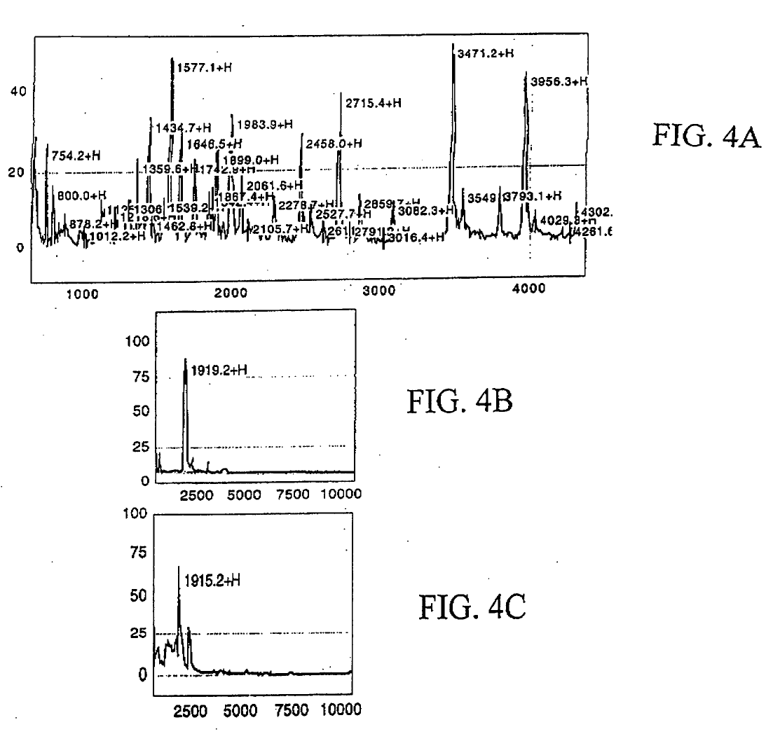

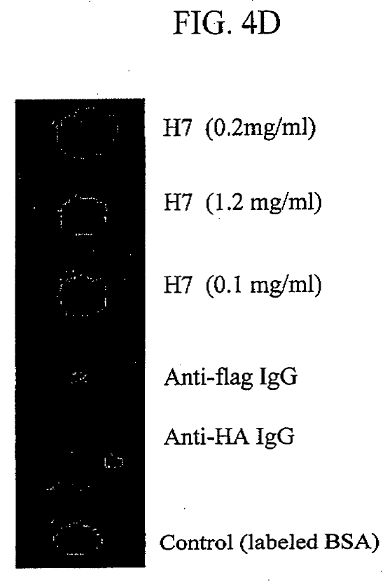

Steady state trypsin cleavage of cell surface proteins was performed on SKBR3 (human breast carcinoma) or SKOV3 cells at 4° C. using TPCK-treated trypsin. Tryptic digests were examined using MALDI mass spectrometry, which is shown in FIG. 4A for SKOV3 cells. About 0.5 μl of the digest was loaded onto a MALDI surface and embedded with matrix consisting of cinnamic acid saturated 50% acetonitryl, 0.5% Triflour, and acetic acid. Digests were treated with protease inhibitors and incubated with 1 μg of purified 6×His-scFv against the transferrin receptor ecto-domain. The scFv-peptide complex was purified from the digests using Ni-NTA sepharose beads. The beads were washed and then were embedded in cinnamic acid matrix as described above. The matrix eluted peptides were analyzed for mass spectrometry, as shown in FIG. 4B. The epitope containing tryptic peptide was identified using the pepident program from the EXPASY suite. For the control experiment HA-tagged transferrin receptor expressed in CHO cells was immuno-precipitated using anti-HA IgG coupled to sepharose beads. The purified protein was displaced from the beads using HA-peptide and then digested with immobilized TPCK-treated trypsin. The scFv epitope-containing peptide was purified using the H7 scFv and analyzed for mass as above and is shown in FIG. 4C. The transfected transferrin protein contain an HA epitope sequence on it's amino terminal (intracellular domain). This tag serves as a control for extracellular-specific labeling.

Trypsin digests of the purified transferrin receptor and of the cell surface proteins were labeled with the primary amine reactive dye NHS-CY-5 and dialyzed against PBS. The labeled peptides were then diluted to a concentration of 0.2 mg/ml in PBS with 10 mg/ml BSA and 0.05% Tween 20 and incubated on the surfaces of glass slides which had been derivatized with the scFv against the transferrin receptor (H7). Incubations were performed overnight in a humidified chamber at 4° C. Binding of CY-5 labeled peptide was determined using a fluorescence scanner. FIG. 4D shows the result of the experiment where the transferrin receptors are shown to bind to the H7 scFv of varying concentrations. Because the HA epitope was on an intracellular domain, the anti-HA IgG serves a negative control here.

(ii) Functionality Testing of scFv Coupled to Maleimide-Derivatized Glass Slides

Spots on a maleimide-derivatized slide surface were outlined with a hydrophobic pen to keep samples from spreading and 1.0 μg of scFv reduced as described in Example C (ii) was then allowed to couple to the glass surfaces for 12 hours at 4° C. in a humidity chamber. The thiol-containing terminal cysteines readily attach to the maleimide groups, presumably by a thioether linkage. Monoclonal antibodies to cytochrome-c and Bcl-2, and scFv without terminal cysteines were treated with 2-iminothiolane•HCl (Traut's reagent) to introduce sulfhydryl residues at surface-exposed lysines. These antibodies were then reduced as described above and used as controls. After coupling, the spots were rinsed 3× with PBS containing 2% BSA, 0.05% Tween 20, and 1.0 mM β-mercaptoethanol for 15 minutes at 25° C. Cognate ligand or negative control were added to the appropriate spots at concentrations ranging from 10.0 pM to 0.01 pM in PBS containing 2% BSA, 0.05%, Tween-20 and allowed to incubate for 2 hours in a humidity chamber at 4° C.

In some cases, 40% glycerol is added to the spotting mixture to facilitate the microarraying of the scFv's, because the samples will not dry out even when spotted in sub-microliter volumes. For scFv C6.5 and scFv F5, 40% glycerol had no adverse effect on the function of the scFv binding.

The cognate ligand for scFvC6.5 is the purified erbB-2 receptor. The recombinant ectodomain of erbB-2 was expresssed and purified from CHO cells using standard techniques. NHS-CY5 monofunctional dye (AMERSHAM) was used to label the protein at a final molar dye/protein ratio of 5.0. The labeling reaction was carried out in 0.1 M sodium carbonate buffer for 30 minutes at 25° C. and exchanged into PBS using a P10 spin column. Other proteins used as controls (Bcl-2, cytochrome-c, and BSA) were similarly labeled with CY5 as described. Labeled proteins were examined for immunogenicity by immuno-precipitation either with phage generated antibody or monoclonal antibodies and were then used as ligands to glass coupled scFv. The erbB-2 proteins were incubated in a range of 1 uM to 1 pM in PBS Tween 20 with 2% BSA for 2 hours at 25° C. in a humidity chamber. CY5 labeled erbB2 was used as a negative control.

After incubation, samples were washed 3×2 minutes with PBS, 0.05% Tween 20 and 1× with PBS. Samples were allowed to dry and then imaged on a molecular dynamics STORM using the excitation at 640 nm.

(iii) Small Molecules in Signal Transduction

Recombinant fusion proteins from the Bcl-2 family of apoptosis regulating proteins were prepared by standard methods and printed on either BSA-NHS glass slides or an aldehyde derivatised glass slide. Proteins were printed at concentrations ranging from 200 to 20 micrograms per milliliter in a buffer containing 40% glycerol. Printing was performed as described using the GMS 417 ring and pin printer. Plates were loaded with the capture protein samples; 96 well plates for printing with the GMS417 printer. Proteins were allowed to incubate on the reactive slides for 12 hours under slightly hydrated conditions at 4° C. After the binding reaction went to completion the slides were rinsed with PBS and variations of the cognate ligand labeled with fluorescent dyes. Detection was performed using the Arrayworx optical reader.

The printed proteins were GST fusions of Bcl-XL and BAX and a 6× histidine-tagged-Bcl-XL. Ligands for these proteins were the full length Bcl-XL protein and the BH3 containing peptide from the Bcl-2 family protein BAK. The peptides were labeled with Alexa 488 and the full length protein was labeled with CY5. The volume of liquid delivered from the GMS printer is 50-70 pL per stroke repeated 5 times. Protein delivered ranged from 350 pg to 350 fg of protein per spot. After printing, proteins were allowed to incubate for 12 hours at 4 degree in a humidity chamber. The slides were then washed with PBS and blocked with PBS with 10% BSA for 5 minutes. To determine the reactivity of the surfaces and the coupling efficiency of the proteins, the presence of the GST-fusion proteins were monitored using labeled anti-GST-tag antibody at 1 ng/ml.

Labeled protein ligands were incubated in a volume of 40 μl contained in an area of 1 cm² by a hydrophobic barrier.

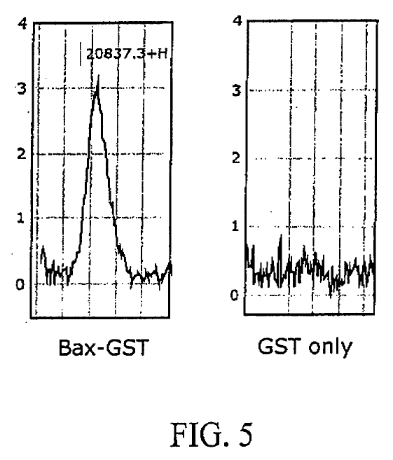

The slides were then rinsed and read using the Arrayworx scanner. In addition, As shown in FIG. 5, which is a mass spectrometry profile, binding of a ligand by a Bax-GST protein is confirmed on the left, while non-binding by a GST protein is shown on the right.

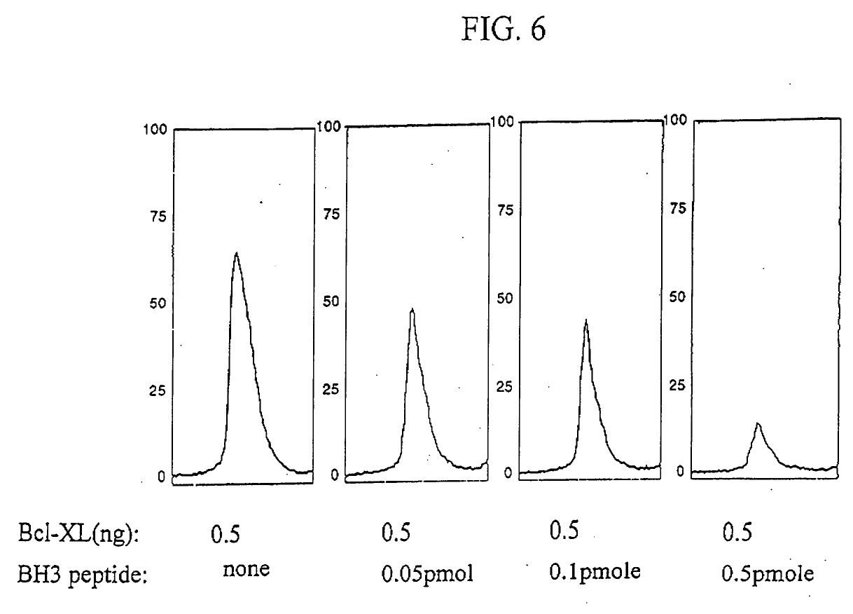

FIG. 6 confirms the ability of an unlabelled small molecule (a BH3 peptide here) to compete a labeled ligand (Bcl-XL here) off the capture molecule (Bax-GST fusion protein). As shown in the four mass spectrometry profiles, with an increasing amount of the BH3 peptide, lesser binding between labeled ligand and the capture protein was observed. This confirmed that the interaction between the capture protein and the ligand was indeed attributable to the BH-3 domain. The same type of experiment was carried out using a small molecule that has been identified as specifically enhancing BH3 protein-protein interaction, and enhancement in ligand (Bcl-XL) binding by a capture molecule (Bak peptide) was observed as expected.

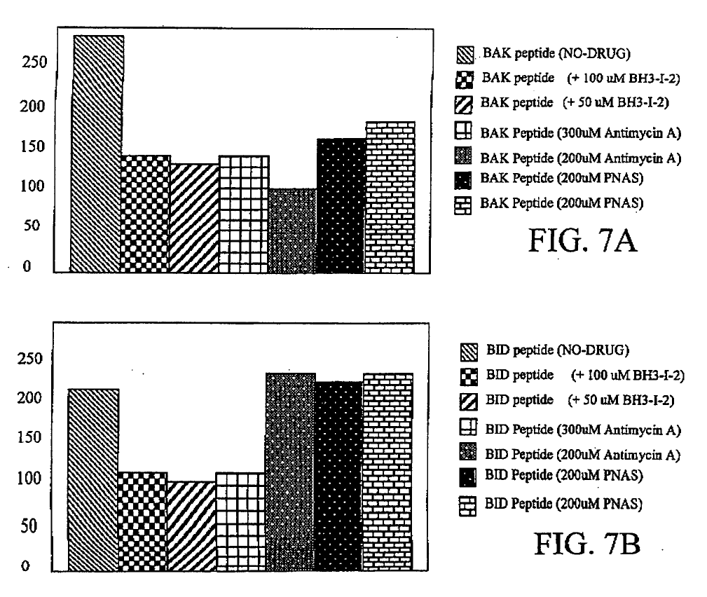

These experiments were then repeated using several peptides of the BH3 family as ligands to compete with three drugs known to affect Bcl-2 family member function at various concentrations. Bcl-XL was printed on BSA-NHS glass slides as capture proteins in each case. The detected fluorescence of the labeled ligand captured on the slide were shown in columns in FIGS. 7A and 7B, different drugs showed differential specificity for the two ligands from the same family. For Bak (FIG. 7A), inhibitory effects were seen in virtually all the cases, while for Bid (FIG. 7B), PNAS or a relatively low concentration of anitmycin does not seem to inhibit its binding. This experiment can be useful in mapping out a drug candidate's specificity regarding each member of a large family of target proteins.

(iv) Cell Surface Protein Expression

Monoclonal and scFv antibodies were printed on glass microarrays for detection of cell surface antigen expression in cancer cell lines. Antibodies to c-ErbB2, EGFR, and transferrin receptor were printed on BSA-NHS activated glass slides. With the monoclonal antibodies, less than 2 ng/mL of recombinant antigen labeled with fluorescent dye was detected. For antigen detection in cell extracts, the cell surfaces of cancer cell lines were labeled with fluorescence using NHS-based dyes. This allowed the detection of differential cell surface expression of c-ErbB2 and EGFR on several cancer cell lines. The transferrin receptor was not detected using the direct labeling approach; however, when a micro-sandwich approach was employed, also the transferrin receptor was detected.

Monoclonal antibodies to c-ErbB2, EGFR, and transferrin receptor (TfR) were arrayed on a GMS 417 arrayer. The antibodies were spotted in 40% glycerol to prevent drying out of the spots onto BSA-NHS slides. Antibodies were allowed to react with the slide overnight in the cold. The resulting spot size was about 150 micrometer with a spacing of 375 micrometer (center to center).

Slides were blocked for 30 minutes in 0.5 M glycine and then in BSA for another 30 minutes before samples were added. When multiple samples were processed on a single slide, groups of antibody spots were separated by drawing with a hydrophobic pen to allow up to 24 samples to be processed per slide. Alternatively, the groups of antibody spots were separated using an adhesive Teflon mask allowing 50 or more samples to be processed per slide.

The samples were usually labeled with Cy3 or Cy5-NHS dyes for one hour at room temperature and un-reacted dye is removed by gel filtration. The cell lines used in this study were the breast adenocarcinoma cell line SKBR3 and the epidermoid carcinoma cell line A-431. Cell surfaces were labeled using the dye, fluorescein-PEG2000-NHS (Shearwater), at 10 mg/mL in PBS for two hours on ice and un-reacted dye was removed by washing the cells before solubilizing in 0.25% SDS in TBS. Recombinant protein antigens were incubated in 2% BSA in 0.1% tween-PBS. Cell lysates were incubated in the lyses buffer without BSA. Following incubation with the samples for two to three hours, the slides were washed 4×10 times: 20 times in TPBS, then 20 times in PBS, by rapid submersion in a beaker containing the wash buffer. The fluorescence was detected using the ArrayWoRx slide reader.

Sensitivity:

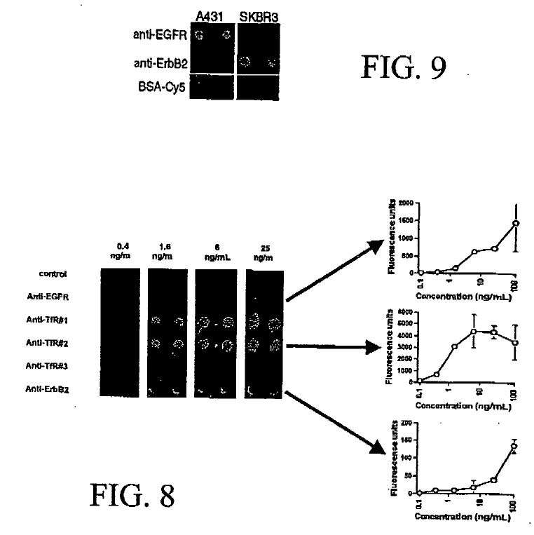

Microarrays were incubated with serial dilutions of ErbB2 labeled with alexa488 and EGFR labeled with Cy5. After washing, the slide was scanned on the ArrayWoRx. As shown in FIG. 8, except for TfR antibody #3, all the antibodies were able to capture ErbB2, TfR, and EGFR respectively. Protein capture was detected at a dilution as low as 1.6 ng/mL.

Detection of Cell Surface Antigens:

The breast adenocarcinoma cell line SKBR3, and the epidermoid carcinoma cell line A-431, were grown to confluence and the cell surface labeled with the dye fluorescein-PEG2000-NHS. Following labeling, un-reacted dye was removed by washing the cells and the cells were lysed in 0.25% SDS. Total labeled protein (corresponding to about 50,000 cells) was then incubated on the antibody microarray for two hours and the slides scanned on the ArrayWoRx. As shown in FIG. 9, the A-431 cell line over-expresses EGFR, but not ErbB2; and the SK-BR-3 cell line over-expresses ErbB2, but only expresses low levels of EGFR. This differential expression of the two receptors in the two cell lines is confirmed by by flow cytometry (e.g., >10⁶ EGFR receptors per cell in A-431 cells).

In a different approach, the cell proteins were not labeled directly with fluorescence. Instead, instead, antigen binding to the array was detected with a second fluorescent-labeled antibody to the antigen. The sensitivity of this “sandwich” detection approach was similar to what was observed for the directly labeled recombinant antigens.

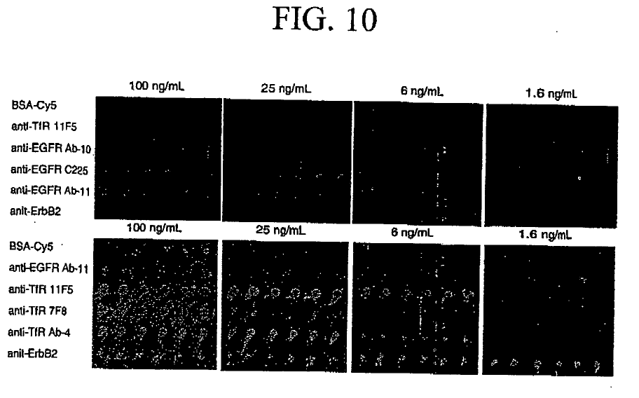

In one experiment, antibodies were printed as before in microarrays and incubated with unlabeled antigens for two hours. Binding was detected with a second antibody to the antigen labeled with Cy5 (for detecting EGFR) or Cy3 (for detecting TfR). Results are shown in FIG. 10: monoclonal antibodies as listed in the legend exhibits good sensitivity at about 25 ng/mL.

The same sandwich approach was performed using phage displayed antibody such as scFv F5 labeled with Cy5.

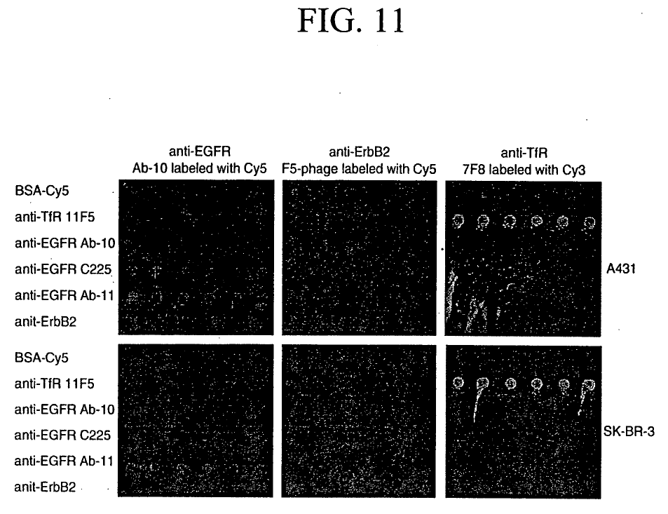

For detection of antigens in cell extracts, cell lines (A431 or SKBR-3) were lysed in 0.25% SDS and extracts were incubated with the antibody array for two hours. After washing, bound antigen was detected with fluorescent monoclonal antibodies (for EGFR and TfR) or phage antibody (for ErbB2). As shown in FIG. 11, using the sandwich approach, all three antigens, EGFR, ErbB2, or TfR, were detected in both cell lysates. The anti-EGFR antibodies detected the differential expression of ErbB2 in the A431 and SK-BR-3 cell lines (>10 fold difference). Like wise, the anti-ErbB2 phage antibody detected the difference in expression of ErbB2 in the two cell lines. As expected, in the case of transferrin receptor expression, no major difference in expression was detected between the two cell lines.

All documents, patents, publications cited above in the specification are herein incorporated by reference. Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the invention.

What is claimed is:

1-20. (canceled)

21. A method for identifying a small molecule regulator of protein binding, the method comprising the steps of:
(a) attaching a capture protein on a support surface;
(b) exposing said substrate to a ligand for said capture protein and at least one small molecule; and
(c) detecting the presence or the absence of binding between said capture protein and said ligand.

22. The method of claim 21, wherein step (a) comprises attaching said capture protein on a BSA-NHS slide.

23. The method of claim 21, wherein step (a) comprises functionalizing said support surface with aldehyde groups.

24. The method of claim 21, wherein step (a) comprises attaching said capture protein in a microarray of at least 1,000 spots per cm².

25. The method of claim 21, further comprising fusing said capture protein to a GST protein.

26. The method of claim 21, further comprising detecting said binding between said capture protein and said ligand through a fluorescent dye.

27. The method of claim 26, wherein said fluorescent dye comprises a hydrophilic polymer moiety.

28. The method of claim 27, wherein said moiety is a polyethyleneglycol.

29. The method of claim 21, wherein step (c) comprises detecting said binding between said capture protein and said ligand through a labeled phage particle displaying an antibody fragment.

30. The method of claim 21, wherein said ligand comprises a family of related proteins.

31. The method of claim 30, wherein said ligand comprises the Bcl-2 family of proteins.

32. The method of claim 21, wherein said capture protein comprises a family of related proteins.

33. A method for identifying a small molecule that selectively affects a cellular pathway, the method comprising the steps of:
(a) attaching a microarray of capture proteins on a support surface, said microarray comprises proteins that act in a cellular pathway;
(b) exposing said substrate surface to at least one ligand of said capture proteins and at least one small molecule; and
(c) detecting a change in binding between said capture proteins and said ligand, said change resulting from interaction with said small molecule.

34. The method of claim 33, wherein step (c) further comprises using mass spectrometry to quantify said change.

35. The method of claim 33, further comprising detecting said binding between said capture protein and said ligand through a fluorescent dye.

36. The method of claim 35, wherein said fluorescent dye comprises a hydrophilic polymer moiety.

37. The method of claim 36, wherein said moiety is a polyethyleneglycol.

38. The method of claim 33, wherein step (c) comprises detecting said binding between said capture protein and said ligand through a labeled phage particle displaying an antibody fragment.

39. The method of claim 33, wherein step (a) comprises attaching said capture proteins on a BSA-NHS slide.

40. The method of claim 34, wherein step (a) comprises attaching said capture protein in a microarray of at least 1,000 spots per cm².

41-72. (canceled)
